(19) United States
(12) Patent Application Publication
(10) Pub. No.: US 2014/0230523 A1
(43) Pub. Date: Aug. 21, 2014

(54) DEVICE AND METHOD FOR MEASURING THE PARTICLE CONCENTRATION IN AN AEROSOL

(71) Applicants: Karl Stengel, Deizisau (DE); Gerhard Haaga, Ohmden (DE); Michael Neuendorf, Plochingen (DE); Joerg Staib, Boll (DE); Andrea Matteucci, Esslingen (DE)

(72) Inventors: Karl Stengel, Deizisau (DE); Gerhard Haaga, Ohmden (DE); Michael Neuendorf, Plochingen (DE); Joerg Staib, Boll (DE); Andrea Matteucci, Esslingen (DE)

(21) Appl. No.: 14/234,049

(22) PCT Filed: Jun. 14, 2012
(86) PCT No.: PCT/EP2012/061323

§ 371 Date: Apr. 23, 2014

(30) Foreign Application Priority Data
Jul. 25, 2011 (DE) 102011079769.6

(51) Int. Cl. G01N 21/53; G01N 33/00

(52) U.S. Cl. CPC G01N 21/53; G01N 33/0009
USPC 73/28.01

(57) ABSTRACT

A device for measuring the particle concentration in an aerosol using a flow tube includes a cavity in which there is a sleeve, the cavity branching off from the flow tube. The sleeve includes, at its end facing away from the flow tube, a collar. The collar encircles the periphery of the sleeve and is fastened at the periphery of the cavity. There is one or more inflow openings in the collar. At least one outflow opening is in an end of the sleeve, which end extends into the flow tube. A measuring chamber is also included in the cavity on the side of the sleeve facing away from the flow tube.

### FIELD OF THE INVENTION

The present invention relates to a device and a method for measuring the particle concentration in an aerosol.

### BACKGROUND

The use of scattered light methods for measuring the concentration of particles in exhaust gases and other aerosols is known.

In this context, a light source, such as a laser, situated in or on a measuring chamber, is usually used, and the aerosol to be measured is guided through the measuring chamber. In or on the measuring chamber, there is at least one light sensor which detects light that has been scattered by the particles present in the aerosol.

In order to ensure durably correct measuring results, the light output surfaces of the light source and the light input surfaces of the light sensors, which come into contact with the aerosol, must be kept free of deposits and condensed water. For this purpose, clean air, in the form of so-called scavenging air curtains, is usually guided over the light input and output surfaces.

This requires an additional expenditure in the construction and operation of the device.

### SUMMARY

Example embodiments of the present invention provide a simplified device and a simplified method for measuring the particle concentration on an aerosol, which will continuously supply correct measuring results, even at longer operation.

For example, according to an example embodiment, a device for measuring the particle concentration in an aerosol includes a flow tube through which the aerosol to be measured flows, and a measuring chamber that is designed to measure the particle concentration in the aerosol. The device also includes a cavity that branches from the flow tube, and a sleeve situated in the cavity which extends, at its first end facing the flow tube, into the flow tube.

At a second end, facing away from the flow tube, the sleeve includes a collar running around the periphery of the sleeve, which is fastened on the periphery of the cavity, with at least one inflow opening developed in the collar. At the first end of the sleeve, which is situated in the flow tube, at least one outflow opening is developed. The measuring chamber is developed on the side of the sleeve in the cavity that faces away from the flow tube.

The exhaust gas flow flowing past the outflow opening of the sleeve generates an underpressure, causing a part of the aerosol flowing through the flow tube to be sucked into the sleeve through the at least one inflow opening, developed in the collar of the sleeve, and flow back again into the flow tube at its end facing the flow tube. A secondary aerosol flow is created through the outer region of the cavity, in the radial direction, the measuring chamber developed in the cavity on the side of the sleeve facing away from the flow tube, and the inside of the sleeve. The continual secondary flow prevents the walls of the measuring chamber from being soiled by deposits and the measuring result from being corrupted. Consequently, a device according to an example embodiment of the present invention provides durably reliable measuring results, even at longer operation. The sleeve also protects the measuring chamber from condensed water that is contained in the aerosol or that condenses from it.

This sleeve according to the present invention is a simple mechanical component which is able to be produced cost-effectively and requires no maintenance in operation.

Example embodiments of the present invention provide a cost-effective device for measuring the particle concentration in an aerosol, which durably and reliably supplies correct measuring results.

In an example embodiment, the outflow opening is developed in an end face of the sleeve facing the exhaust branch. Through the outflow opening, the flow in the flow tube develops a particularly good suction effect and a great pressure drop on the inside of the sleeve.

In another example embodiment, the sleeve is a conventional protective cap as is used to protect lambda probes. Protective caps for lambda probes are produced in large numbers at low cost, and provide easy-to-procure and cost-effective sleeves which are well suitable for use in the device according to the present invention.

In an example embodiment, the device includes at least one light source and at least one light sensor. A light source and a light sensor make it possible to determine the particle concentration in the aerosol with the aid of irradiated light and particularly by a scattered light measurement.

In an example embodiment, the measuring chamber includes transparent windows, which enable light to radiate through the measuring chamber. This makes it possible to position the light source and the light sensor outside the measuring chamber.

In an example embodiment, the measuring chamber is developed as a scattered light measuring chamber, the light sensor records the light (scattered light) scattered by the particles present in the aerosol in the measuring chamber, and the concentration of particles in the aerosol is determined from the intensity of the scattered light. Scattered light measuring chambers represent a proven way for determining the particle concentration in aerosols.

In one example embodiment, the cavity is closed on the side facing away from the flow tube by a removable stopper. A removable stopper enables access to the measuring chamber and/or to the sleeve, for the purpose of maintaining the latter as needed and/or exchanging it.

In one example embodiment, the removable stopper is screwed into the cavity. By screwing the stopper into the cavity it is ensured that the stopper is reliably fixed and that it seals the cavity in a gas-tight manner.

In an example embodiment, the cavity is developed at essentially a right angle to the longitudinal extension of the flow tube. A cavity developed at a right angle to the longitudinal extension of the flow tube is easy to produce, and enables a good secondary flow through the measuring chamber and the sleeve.

In one example embodiment, the cavity is developed to be cylindrical. A cylindrical cavity is produced in a particularly simple and cost-effective manner.

In an example embodiment, the sleeve is situated at essentially a right angle to the longitudinal extension of the flow tube. At an orientation at a right angle to the longitudinal extension of the flow tube, the sleeve is particularly easy to mount, and the exhaust gas flow flowing past the sleeve in the flow tube generates a particularly high underpressure in the sleeve.

According to an example embodiment of the present invention, a method for measuring the particle concentration in an aerosol includes guiding the aerosol through a device as described herein.

In the following text, example embodiments of the present invention will be explained in greater detail with reference to the appended figures.

### BRIEF DESCRIPTION OF THE DRAWINGS

FIG. 1 provides a schematic view of a device according to an example embodiment of the present invention.

FIG. 2 shows an enlarged section of a device according to an example embodiment of the present invention.

### DETAILED DESCRIPTION

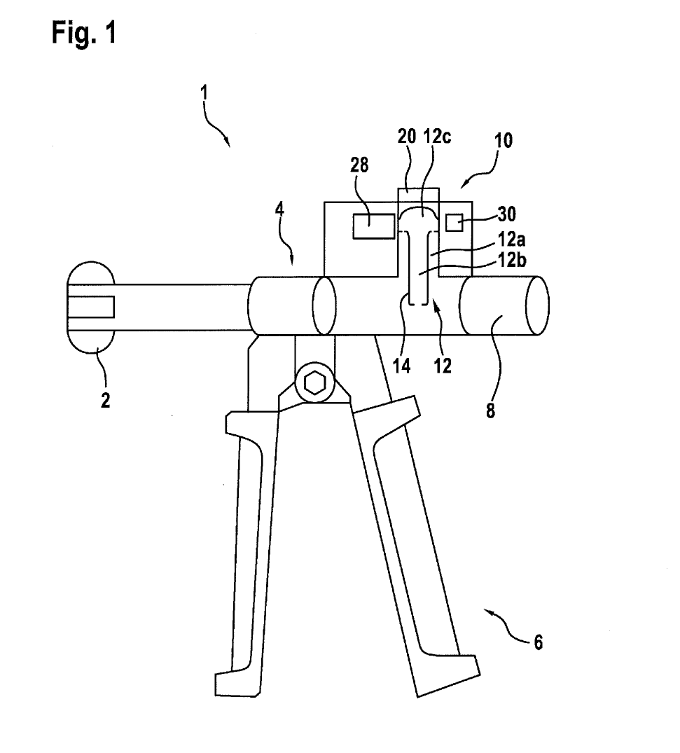

FIG. 1 shows a schematic side view of an example embodiment of a device 1 according to the present invention. Device 1 includes a flow tube 4. The flow tube 4 includes an end 2 at an input side and an end 8 at an output side. For the measurement of the particle concentration, end 2 of flow tube 4, at the input side, is positioned in such a way in the flow tube of an internal combustion engine that the aerosol to be measured (e.g., the exhaust gases to be measured) enters flow tube 4 at the input-side end 2, flows through flow tube 4 and exits from flow tube 4 through end 8 at the output side. At end 8, a hose or another accommodation device can be mounted to take up the aerosol exiting from flow tube 4 and guide it away.

A clamp or handle 6 is mounted on flow tube 4 to make it possible to position flow tube 4 simply and conveniently in the desired position in or on the exhaust branch.

A measuring device 10, according to the present invention, is also mounted, which makes it possible to measure the concentration of particles, contained in the aerosol which flows through flow tube 4.

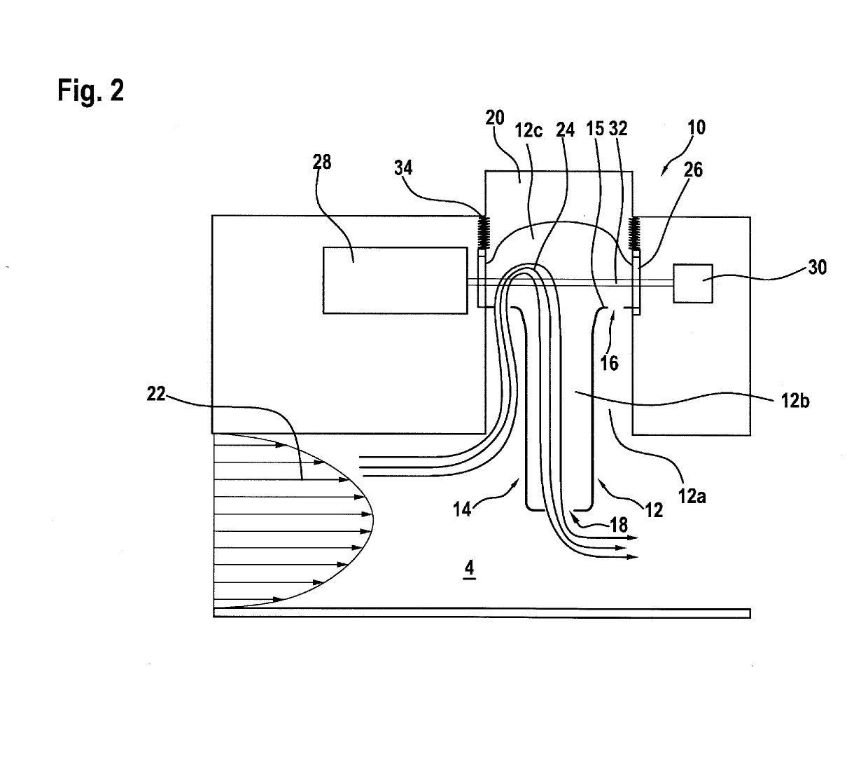

The construction and the function of a measuring device 10 according to the example embodiment shown in the figures is described below with the aid of an enlarged representation as shown in FIG. 2.

FIG. 2 shows an enlarged representation of a measuring device 10, according to an example embodiment of the present invention, which is mounted on a flow tube 4.

The illustrated measuring device 10 includes a cavity 12, which branches from flow tube 4 and is in flow connection with flow tube 4. In the example embodiment shown in FIGS. 1 and 2, cavity 12 is developed to be cylindrical, the axis of the cylinder being situated at a right angle to the longitudinal extension of flow tube 4.

Cavity 12 is closed by a stopper 20 on the side shown at the top in FIG. 2, facing away from flow tube 4, which is fixed in cavity 12 by a screw joint 34. Stopper 20 may be made of rubber, for example, or another elastic material

A sleeve 14 is situated along the longitudinal axis of cylindrical cavity 12. Sleeve 14 is developed to be pot-shaped, and is situated with its longitudinal axis essentially parallel to the longitudinal extension of cavity 12 at a right angle to the longitudinal extension of flow tube 4, and, therefore, also to flow 22 in exhaust pipe 4. Sleeve 14, in this context, extends with its lower end from the lower end of cavity 12, facing flow tube 4, into flow tube 4, so that the lower end of sleeve 14 is situated within flow tube 4 with aerosol flow 22 flowing around it in flow tube 4.

There is an outflow opening 18 in the end face of sleeve 14 facing flow tube 4.

At the opposite end of sleeve 14, facing away from flow tube 4, sleeve 14 includes a collar 15 that encircles the periphery of sleeve 14, and which is fixed to the wall that limits the periphery of cavity 12, and thus fastens sleeve 14 in cavity 12.

Outflow openings 16 in collar 15 create a flow connection between an outer region 12a, in the radial direction, of cavity 12, which is situated around the periphery of sleeve 14, and a region 12c of cavity 12 above the collar.

Region 12c of cavity 12 above sleeve 14 is developed as a measuring chamber that includes two measuring windows 26, through which, during operation, a light beam 32, generated by a light source 28, e.g., a laser light source, is beamed through measuring chamber 12c. Light beam 32 exiting from measuring chamber 12c or the light scattered by the particles contained in the aerosol, exits through a second window 26 from the measuring chamber and is detected by at least one light sensor 30. The signal emitted by the at least one light sensor 30 is supplied to an evaluation device that is not shown in FIG. 2, in order to determine the particle concentration of the aerosol in measuring chamber 12c.

During operation, the aerosol to be measured flows along the longitudinal extension of, and through, flow tube 4. In the process, flow 22 generates an underpressure at outflow opening 18 of sleeve 14 on the flow tube side, which causes a flow from the inside 12b of sleeve 14 into flow tube 4. On the inside 12b of sleeve 14 an underpressure is created, which leads to an after-surge of aerosol from flow tube 4 through inflow openings 16, that are developed in collar 15 of sleeve 14, into measuring chamber 12c and from there into the interior 12b of sleeve 14. A secondary flow 24 is created through the outer region 12a of cavity 12 (which is developed around the periphery of sleeve 14) measuring chamber 12c, and the interior of sleeve 14.

Measuring chamber 12c and particularly windows 26 of measuring chamber 12c are protected, in this instance, from condensed water, which may be contained in aerosol 22.

In the example embodiment shown in the figures, a pot-shaped sleeve 14 is used, but other shapes can be used instead. Sleeve 14 can be of an arbitrary shape, as long as its openings 16, 18 are developed and situated in such a way that they enable a secondary flow 24 through measuring chamber 12c, and the pressure difference required to effect secondary flow 24 at sleeve 14 is produced.

The design of a device according to the present invention effects a continuous flow 24 of the aerosol via windows 26 of measuring chamber 12c, so that deposits of soot or other dirt particles are reliably avoided on windows 26 of measuring chamber 12c, which could falsify the measuring results.

Compared to the usual design approaches, which use a scavenging air curtain in order to keep the measuring chamber free from deposits, a device according to the present invention is simpler, smaller, and more cost-effective to implement. In particular, it is simpler to implement using cost-effective components, such as sleeves that are used for lambda sensors. A device according to the present invention can also be integrated without a problem into the usual probes, such as the ones used for exhaust gas measurement.

1-10. (canceled)

11. A device for measuring a particle concentration in an aerosol, comprising:
a flow tube;
a cavity branching off from the flow tube;
a sleeve in the cavity, the sleeve including:
a first end that extends into the flow tube and at which there is at least one outflow opening;
a second end that faces away from the flow tube; and
a collar at the second end that:
encircles a periphery of the sleeve;
is fastened at a periphery of the cavity; and
includes at least one inflow opening; and
a measuring chamber in the cavity at a side of the sleeve facing away from the flow tube.

12. The device of claim 11, wherein the outflow opening is in an end face of the sleeve facing the flow tube.

13. The device of claim 11, further comprising at least one light source and a light sensor.

14. The device of claim 11, wherein the sleeve is a protective cap of a lambda probe.

15. The device of claim 11, wherein the measuring chamber includes transparent windows which enable light to enter into or exit from the measuring chamber.

16. The device of claim 11, wherein the cavity is closed by a removable stopper at a side of the cavity facing away from the flow tube.

17. The device of claim 16, wherein the stopper is screwed into the cavity.

18. The device of claim 11, wherein the cavity is at approximately a right angle to a longitudinal extension of the flow tube.

19. The device of claim 11, wherein the sleeve is at approximately a right angle to a longitudinal extension of the flow tube.

20. A method for measuring a particle concentration in an aerosol, comprising:
conducting the aerosol through a device, the device including:
a flow tube;
a cavity branching off from the flow tube;
a sleeve in the cavity, the sleeve including:
a first end that extends into the flow tube and at which there is at least one outflow opening;
a second end that faces away from the flow tube; and
a collar at the second end that:
encircles a periphery of the sleeve;
is fastened at a periphery of the cavity; and
includes at least one inflow opening; and
a measuring chamber in the cavity at a side of the sleeve facing away from the flow tube.
